(19) United States
(12) Patent Application Publication
(10) Pub. No.: US 2026/0078331 A1
(43) Pub. Date: Mar. 19, 2026

(54) BACTERIAL INSPECTION APPARATUS AND BACTERIAL INSPECTION METHOD

(71) Applicant: SHIMADZU CORPORATION, Kyoto-shi (JP)

(72) Inventors: Junichi OHI, Kyoto-shi (JP); Shuhei MATSUDA, Kyoto-shi (JP)

(21) Appl. No.: 19/328,905

(22) Filed: Sep. 15, 2025

(30) Foreign Application Priority Data
Sep. 18, 2024 (JP) 2024-160726

(51) Int. Cl. C12M 1/34; C12M 1/00; C12M 1/12; C12M 1/32; C12M 3/00

(52) U.S. Cl. CPC C12M 41/36; C12M 23/12; C12M 23/34; C12M 23/38; C12M 23/48; C12M 29/04; C12M 37/00

(57) ABSTRACT

A bacterial inspection apparatus 1 comprises one or more containers 2 for storing a bacterial solution 23 containing bacteria 7 and a culture medium, a sensor 22 provided at a bottom part 21 of each of the one or more containers 2 and having a detection region S for the bacteria 7 above a top surface thereof, a cover 32 for sealing the bacteria 7, and a spacer 26 for supporting the cover 32 in a non-contact state with the top surface of the sensor 22.

### TECHNICAL FIELD

The present invention relates to a bacterial inspection apparatus and a bacterial inspection method.

### BACKGROUND ART

As a conventional bacterial inspection apparatus, there is one that detects the presence of bacteria, such as live bacteria like Mycobacterium tuberculosis and Escherichia coli. Such bacterial inspection apparatuses include those that detect bacteria using a CMOS (Complementary Metal-Oxide-Semiconductor) oscillator sensor.

As an example of a conventional bacterial inspection apparatus using a CMOS oscillator sensor, Non-Patent Literature 1 discloses an apparatus that detects the presence of bacteria in a bacterial solution by measuring the dielectric constant of the bacterial solution using a sensor, based on the principle that the dielectric constant of the bacterial solution changes as bacteria multiply within the bacterial solution, which contains bacteria and a culture medium.

### CITATION LIST

### Non-Patent Literature

[Non-Patent Literature 1] Y. Ogawa et al., “Near-field sensor array with 65-GHz CMOS oscillators for rapid detection of viable Escherichia coli”, Biosensors and Bioelectronics, volume 176, 15 Mar. 2021, https://doi.org/10.1016/j.bios.2020.112935.

### SUMMARY OF THE INVENTION

### Technical Problem

When a sensor detects bacteria in a bacterial solution by measuring the dielectric constant of the bacterial solution, the following problem may arise. Hereinafter in this specification, the indirect detection of bacteria by a sensor measuring the dielectric constant or the like will also be described as “a sensor detects bacteria.” For example, in a state where the depth of a bacterial solution stored in a reservoir, such as a well, exceeds the detection region of the sensor in the depth direction of the reservoir, the sensor cannot detect bacteria in the bacterial solution outside the detection region. This makes it difficult for the sensor to reliably detect bacteria in the bacterial solution. Therefore, to solve this problem, it is conceivable to provide a filter at one end of a housing member inserted into the upper opening of the reservoir and attach a weight to the housing member, thereby lowering the housing member within the internal space of the reservoir to bring the bacteria within the sensor's detection region and reliably detect the bacteria. In this case, the filter and the sensor come into contact as the housing member descends within the internal space of the reservoir, but this could apply a large and non-uniform pressure to the top surface of the sensor, potentially affecting the sensor's detection values.

This invention was made to solve such a problem, and its objective is to enable a sensor to accurately detect the state of bacteria in a bacterial solution.

### Solution to Problem

A bacterial inspection apparatus according to a first aspect of the present invention comprises one or more containers for storing a bacterial solution containing bacteria and a culture medium, a sensor provided at the bottom of each of the one or more containers and having a bacteria detection region above its top surface, a cover for sealing the bacteria, and a spacer for supporting the cover in a non-contact state with the top surface of the sensor.

A bacterial inspection apparatus according to a second aspect of the present invention comprises a container for storing a bacterial solution containing bacteria and a culture medium, a sensor provided at the bottom of each container and having a bacteria detection region above its top surface, and a cover for sealing the bacteria, wherein the cover is formed of a material that has a specific gravity greater than the culture medium and does not affect the detection of bacteria by the sensor.

A bacterial inspection method according to a third aspect of the present invention includes a step of storing a bacterial solution containing bacteria and a culture medium in a container having a sensor provided at its bottom, the sensor having a bacteria detection region above its top surface, and a step of sinking a cover into the bacterial solution to seal the bacteria with the cover and a spacer that supports the cover in a non-contact state with the top surface of the sensor.

A bacterial inspection method according to a fourth aspect of the present invention includes a step of storing a bacterial solution containing bacteria and a culture medium in a container having a sensor provided at its bottom, the sensor having a bacteria detection region above its top surface, and a step of sinking a cover into the bacterial solution to seal the bacteria, wherein the cover is formed of a material that has a specific gravity greater than the culture medium and does not affect the detection of bacteria by the sensor.

### Advantageous Effects of Invention

The state of bacteria in a bacterial solution can be accurately detected.

### BRIEF DESCRIPTION OF DRAWINGS

FIG. 1 is a diagram showing the configuration of a bacterial inspection apparatus of Embodiment 1.

FIG. 2 is a cross-sectional view showing the operational state of a container and a cover during a bacterial inspection by the bacterial inspection apparatus of Embodiment 1.

FIG. 3 is a diagram showing a configuration in which a spacer is arranged on the side surface of a container.

FIG. 4 is a diagram showing a configuration in which a spacer is arranged on the lower surface of a cover.

FIG. 5 is a diagram showing a configuration in which a container is formed in a tapered shape.

FIG. 6 is a diagram showing a configuration using a low-rigidity cover.

FIG. 7 is a flowchart showing the processing procedure of a bacterial inspection method.

FIG. 8 is a diagram showing the configuration and operational state of a bacterial inspection apparatus of Embodiment 2.

FIG. 9 is a diagram showing the configuration and operational state of the bacterial inspection apparatus of Embodiment 2.

FIG. 10 is a diagram showing the configuration and operational state of a bacterial inspection apparatus of Embodiment 3.

FIG. 11 is a diagram showing the configuration and operational state of a bacterial inspection apparatus of Embodiment 4.

FIG. 12 is a diagram showing the structure of a multi-well cover, a support member, and a cover of Embodiment 4.

FIG. 13 is a graph showing the operational state of all elements of a representative sensor when a bacterial solution containing Bacille Calmette-Guerin (hereinafter BCG) is stored in a container during a bacterial inspection by a conventional bacterial inspection apparatus.

FIG. 14 is a two-dimensional map image showing the operational state of all elements of a representative sensor when a bacterial solution containing BCG is stored in a container during a bacterial inspection by a conventional bacterial inspection apparatus.

FIG. 15 is a graph showing the operational state of all elements of a representative sensor when a bacterial solution containing BCG is stored in a container during a bacterial inspection by the bacterial inspection apparatus of the present method.

FIG. 16 is a two-dimensional map image showing the operational state of all elements of a representative sensor when a bacterial solution containing BCG is stored in a container during a bacterial inspection by the bacterial inspection apparatus of the present method.

FIG. 17 is a graph showing the operational state of all elements of a representative sensor when a culture medium not containing BCG is stored in a container during a bacterial inspection by the bacterial inspection apparatus of the present method.

FIG. 18 is a two-dimensional map image showing the operational state of all elements of a representative sensor when a culture medium not containing BCG is stored in a container during a bacterial inspection by the bacterial inspection apparatus of the present method.

FIG. 19 is a graph showing the percentage of sensor elements for which the resonance frequency increased by 1 MHz or more with respect to the measurement time in the graph shown in FIG. 15.

FIG. 20 is a histogram of the resonance frequency increase values of all elements after a predetermined time has elapsed when a culture medium not containing BCG is stored in the container, in the graph shown in FIG. 17.

FIG. 21 is a histogram of the resonance frequency increase values of all elements after a predetermined time has elapsed when a bacterial solution containing BCG is stored in the container, in the graph shown in FIG. 15.

FIG. 22 is a graph showing the change over time of the standard deviation (S.D.) of ΔF in the diagrams shown in FIG. 15 and FIG. 17.

### DESCRIPTION OF EMBODIMENTS

Hereinafter, embodiments of the present invention will be described in detail with reference to the drawings. In the following, the same or corresponding parts in the drawings are denoted by the same reference signs, and a description thereof will not be repeated in principle. Although multiple embodiments will be described below, it is intended from the time of filing that the configurations described in each embodiment can be combined as appropriate.

In the following embodiments, as a basic configuration, a configuration of a bacterial inspection apparatus 1 will be described, in which a bacterial solution 23 containing bacteria 7 and a culture medium is stored in a container in which a sensor 22 for detecting the bacteria 7 is provided at a bottom part 21, a cover 32 is sunk into the bacterial solution, and the bacteria 7 are sealed by the cover 32 in a sealed space 20 above the sensor 22. With this configuration, the movement of the bacteria 7 due to convection is restricted, and changes in the dielectric constant of the bacterial solution within the detection region due to the growth of the bacteria 7 can be measured accurately, stably, and with good reproducibility.

### Embodiment 1

In Embodiment 1, the configuration of the bacterial inspection apparatus 1 including the above-described basic configuration will be described.

### Overall Configuration of Bacterial Inspection Apparatus 1

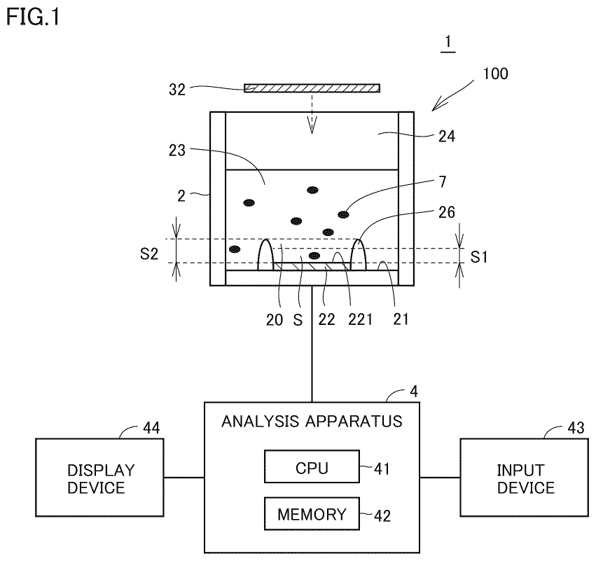

FIG. 1 is a diagram showing the configuration of the bacterial inspection apparatus 1 of Embodiment 1.

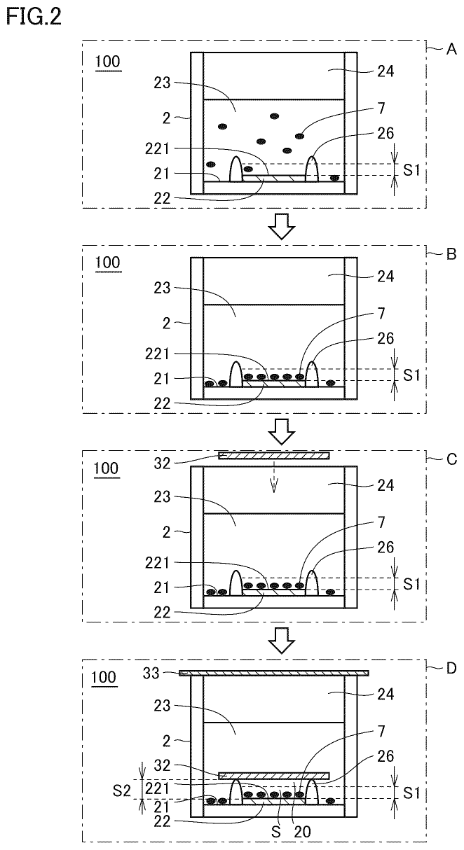

The configuration of the bacterial inspection apparatus 1 will be described with reference to FIG. 1. The bacterial inspection apparatus 1 includes one or more container units 100, an analysis apparatus 4, an input device 43, and a display device 44, respectively. Each of the container units 100 includes a container 2, a sensor 22, a spacer 26, a cover 32, and an evaporation prevention material 33 (see state D in FIG. 2). In FIG. 1, a cross-sectional view of the container unit 100 is shown.

In FIG. 1 and subsequent figures, a plane horizontal to the top surface (bottom surface) of the bottom part 21 of the container 2 is defined as an XY plane, and a direction perpendicular to the bottom surface is defined as a Z direction. In one embodiment, the container 2 during and after storage of the bacterial solution 23 is placed stationary such that the Z direction is approximately the vertical direction (except when sedimenting bacteria by a centrifuge, as described later).

In one embodiment, the container 2 is a cylindrical well, is formed of a transparent resin, has an open top, and has a sealed bottom for storing a bacterial solution 23 containing bacteria 7 and a culture medium.

An example of the size of the bacteria 7 measured by the bacterial inspection apparatus 1 is 0.5 to 10 μm. As a more specific example, in the bacterial inspection apparatus 1, a culture test (hereinafter sometimes referred to as a bacterial inspection) of live bacteria such as Mycobacterium tuberculosis or Escherichia coli is performed. In this case, a bacterial solution 23 containing live bacteria and a culture medium is injected into the container 2 from the upper part of the container 2 with a pipette or the like. In the container 2, the injected bacterial solution 23 is stored in a manner that it accumulates upward from the bottom part 21 along the container 2. The bacterial solution 23 may also contain metabolites of the bacteria 7.

At the bottom part 21 of the container 2, a sensor 22 for detecting the bacteria 7 in the bacterial solution 23 is installed. The sensor 22 may be fixedly attached to the bottom part 21 of the container 2, or it may be detachably attached. In the example of FIG. 1, the sensor 22 is installed on the top surface (bottom surface) of the bottom part 21. In one embodiment, the sensor 22 has a disk shape.

The sensor 22 has a detection region S for the bacteria 7 above its top surface 221. In this specification, “detection region” refers to a region where the bacteria 7 can be detected by the sensor. In one embodiment, the detection region is a region where the dielectric constant can be measured by the sensor 22.

In one embodiment, the sensor 22 is an array sensor in which a large number of elements constituting a resonator are arranged adjacently in a matrix. The elements are, for example, CMOS resonators. By using the sensor 22, the bacteria 7 in the bacterial solution 23 can be detected two-dimensionally based on the fact that the frequency of each element changes (increases) in response to a change (decrease) in the dielectric constant in the bacterial solution 23. Note that, more strictly, since the dielectric constant in the bacterial solution 23 reflects not only the bacteria 7 but also the metabolites of the bacteria 7, the change in the dielectric constant of the bacterial solution 23 in this specification includes the influence of the metabolites of the bacteria 7. Further, “detection of bacteria 7” in this specification is assumed to include the influence of the metabolites of the bacteria 7.

The detection region of each element of the sensor 22 inside the container 2 is, for example, a limited range of several tens of μm to the left, right, and upward (in the height direction) when viewed from the top surface 221 of the sensor 22. The detection region S of the element is also called a sensitivity region, and bacteria 7 outside the detection region S cannot be detected. In FIG. 1, the detection region S corresponds to the space sandwiched by the spacers 26 on the sensor 22 up to a height S1 from the top surface 221 of the sensor 22.

The cover 32 seals the bacteria 7 in a sealed space above the sensor 22 by being sunk into the bacterial solution 23 stored in the container 2 (described later with reference to FIG. 2). Therefore, the outer diameter of the cover 32 is typically configured to be smaller than the inner diameter of the container 2.

In Embodiment 1, the cover 32 is configured as follows. The outer diameter of the cover 32 is configured to be larger than the top surface 221 of the sensor 22. Further, the cover 32 is formed of a material having a specific gravity greater than that of the bacterial solution 23 (more specifically, the specific gravity of the culture medium). As a result, when the cover 32 is placed in the container 2, it sinks naturally, reaches the spacer 26, and forms a sealed space 20 (see D in FIG. 2 described later). Further, in Embodiment 1, the cover 32 is formed of a material that does not affect the components of the bacterial solution 23. This prevents the cover 32 from affecting the growth of the bacteria 7. In one embodiment, the cover 32 is formed of a resin, metal, glass, or the like that satisfies the above two conditions. As a result, it sinks into the bacterial solution 23 without distortion or bending and does not shift due to shaking when the container 2 is moved.

The spacer 26 is a member that forms the sealed space 20. The spacer 26 is a support member that supports the cover 32 in a non-contact state with the top surface 221 of the sensor 22. By providing the spacer 26, the height of the cover 32 from the sensor 22 can be maintained at a predetermined height S2 with a simple configuration, and the sealed space 20 having the predetermined height S2 can be formed. In Embodiment 1, the spacer 26 forms the sealed space 20 between the sensor 22, the cover 32, and the spacer 26 by separating the sensor 22 and the cover 32.

Typically, the diameter of the cover 32 is larger than the diameter of the opening formed by the spacer 26. As a result, the sealed space 20 can be formed with only two simple members, the cover 32 and the spacer 26.

The sealed space 20 is a space where the bacteria 7 are sealed during measurement. This prevents the bacteria 7 from moving in and out of the sealed space 20 during measurement. As a result, changes in the amount of the bacteria 7 and metabolites of the bacteria 7 from the start of the measurement reflect the multiplication by division of the bacteria 7 that were in the sealed space 20 at the start of the measurement, a decrease in living cells, maintenance due to a balance between such multiplication and decrease, cessation of multiplication, and death. Therefore, the state of the bacteria 7 (multiplication, decrease, maintenance, cessation, death) can be analyzed in a state where there is no entry or exit of the bacteria 7 into and out of the sealed space 20.

The sealed space 20 contains a very small portion of the bacterial solution 23 in the container 2. In one embodiment, the sealed space 20 is designed such that convection within the sealed space 20 does not occur, or more precisely, such that convection is so small as to be negligible.

In FIG. 1 and FIG. 2, the spacer 26 is a ring-shaped member arranged at the bottom part 21 of the container 2 so as to surround the sensor 22. In one embodiment, the height S2 of the spacer 26 from the sensor 22 is configured to be higher than the height S1 of the detection region S of the sensor 22. As a result, the cover 32 does not enter the detection region S, so it is not necessary to consider the influence of the material of the cover 32 on the sensor 22. This expands the options for the material of the cover 32.

Detection signals from the sensor 22 are input to the analysis apparatus 4. The analysis apparatus 4 is configured by a computer including a CPU (Central Processing Unit) 41, a memory 42 (various storage devices including ROM (Read Only Memory), RAM (Random Access Memory), and non-volatile memory such as flash memory), and an input/output buffer (not shown) for inputting and outputting various signals.

The CPU 41 expands and executes software programs stored in the ROM into the RAM or the like. The ROM stores various programs that indicate the processing procedures related to analysis by the analysis apparatus 4. The analysis apparatus 4 operates according to such programs and performs analysis related to bacterial inspection in response to the detection signal input from the sensor 22. The analysis related to bacterial inspection is not limited to processing by software, and it can also be processed by dedicated hardware (electronic circuit).

Connected to the analysis apparatus 4 is an input device 43 for inputting various data, such as data used for various analyses related to bacterial inspection, and for inputting operational inputs necessary when executing various analyses related to bacterial inspection. The input device 43 includes various input devices such as a keyboard and a mouse.

Connected to the analysis apparatus 4 is a display device 44 for displaying various images, such as analysis results in a bacterial inspection. The display device 44 includes various display devices such as a liquid crystal display device.

The analysis apparatus 4 analyzes the growth status of the bacteria 7 in the bacterial solution 23 according to the resonance frequency of each element in the array sensor constituting the sensor 22 based on the detection signal input from the sensor 22, and displays the analysis result on the display device 44. Each element in the array sensor can detect the bacteria 7 in the bacterial solution 23 by being resonated at a frequency in the gigahertz band, for example. The frequency for resonating each element in the array sensor is, for example, in the gigahertz band, such as 30 GHz to 300 GHz. It is desirable to use a frequency around 60 GHz for such a frequency.

The analysis apparatus 4 determines that the bacteria 7 have grown by utilizing the fact that the dielectric constant of the bacterial solution 23 changes according to the growth of the bacteria 7 in the detection region S of each element of the sensor 22, and the resonance frequency of each element changes with that change. For example, the analysis apparatus 4 acquires the change in the resonance frequency of each element of the array sensor constituting the sensor 22 in a time series, and determines the growth status of the bacteria 7 in the bacterial solution 23 based on the acquired change in the resonance frequency.

### Operational State of Cover 32 During Bacterial Inspection

Next, the operational state of the cover 32 when a bacterial inspection is performed by the bacterial inspection apparatus 1 will be described. FIG. 2 is a cross-sectional view of the container unit 100 showing the operational state of the cover 32 during a bacterial inspection by the bacterial inspection apparatus 1 of Embodiment 1.

In FIG. 2, a first state A is a state in which the bacterial solution 23 has been injected into the container 2 with a pipette or the like. In FIG. 2, a second state B is a state in which the bacteria 7 have been caused to fall by gravity or have been forcibly dropped to the bottom surface of the container 2 using a centrifuge and have sedimented. In FIG. 2, a third state C is a state before the cover 32 is sunk into the container 2. In FIG. 2, a fourth state D is a state in which the cover 32 placed in the container 2 has sunk by its own weight and rests on the upper part of the spacer 26 formed inside the container 2. In one embodiment, the height S2 of the spacer 26 is sufficiently higher than the height S1 of the detection region S. As a result, the cover 32 does not enter the detection region S of the sensor 22 and does not affect the resonance frequency of the sensor 22.

More specifically, when a bacterial inspection is performed by the bacterial inspection apparatus 1, first, in a state where the cover 32 is not placed inside the container 2 as in the first state A, the bacterial solution 23 containing the bacteria 7 is stored in the internal space 24 of the container 2. In the first state A, the liquid level of the stored bacterial solution 23 is at a position higher than the detection region S of the sensor 22. In this state, the bacteria 7 are dispersed throughout the bacterial solution 23. Therefore, a large number of bacteria 7 in the bacterial solution 23 exist above the detection region S of the sensor 22.

When left to stand for a long time in the first state A to allow the bacteria 7 to fall naturally due to gravity, or when the container unit 100 is placed in a centrifuge to forcibly drop the bacteria 7 to the bottom surface of the container 2 in a short time, the second state B is reached. The long time depends on the depth of the bacterial solution 23 in the container 2, but is, for example, a time between several hours and several days, and more specifically, for example, about two days. The short time is, for example, 10 minutes or less.

Next, as shown in the third state C, when the cover 32 is dropped into the internal space 24 from the upper part of the container 2, the cover 32 passes through the bacterial solution 23 and reaches the upper end of the spacer 26. As a result, a sealed space 20 is formed inside the spacer 26 below the cover 32. When an evaporation prevention material 33 is attached to the upper part of the container 2 in this state, the state of the fourth state D is reached.

In the state of the fourth state D, the bacteria 7 existing within the detection region S of the sensor 22 are detected by the sensor 22.

As described above, the sealed space 20 is formed by the cover 32 covering the area above the sensor 22. Inside the container 2, convection occurs due to the heat generated by the sensor 22 and the temperature difference between the upper and lower parts of the container 2. Further, even when the sealed space 20 is formed, the convection may occur outside the sealed space 20. On the other hand, inside the sealed space 20, because the volume inside the sealed space 20 is small, convection is difficult to occur and is negligible, so the bacteria 7 do not diffuse upward and remain in the detection region S. Then, because the cover 32 and the top surface 221 of the sensor 22 are separated, no pressure is applied to the top surface of the sensor 22. As a result, the frequency change of the sensor 22 due to the growth of the bacteria 7 can be accurately measured. This also improves the reproducibility of the measurement.

The inventors have found the following facts through experiments. When the cover 32 is sunk immediately after injecting the bacterial solution 23 while the bacteria 7 are dispersed in the bacterial solution, a large number of bacteria 7 leak from the gap between the spacer 26 and the cover 32, and the number of bacteria in the detection region S decreases. However, if the cover 32 is sunk after allowing the bacteria 7 to fall naturally by gravity over time, or after forcibly dropping the bacteria 7 by a centrifuge, most or all of the bacteria 7 remain sedimented, and a large number of bacteria 7 did not leak from the gap between the spacer 26 and the cover 32. As described above, it was found that by sealing the sedimented bacteria 7 with the cover 32, a larger number of bacteria 7 can be targeted for measurement, and thus stable measurement can be performed. In addition, the sealed space 20 can be configured to be larger than the detection region S. In particular, according to the configuration in which the bacteria 7 sedimented on the sensor 22 by a centrifuge are sealed by the cover 32, the bacteria 7 can be sedimented in a short time, so the measurement throughput can be further improved.

The size ratios of the various parts such as the size of the container 2, the thickness and area of the sensor 22, the area and height S1 of the detection region S above the sensor 22, the thickness and area of the cover 32, the size of the spacer 26, and the size of the bacteria 7 in FIG. 1 and FIG. 2 are different from the actual size ratios in order to show the technical features of the bacterial inspection apparatus 1 in an easy-to-understand manner.

The actual sizes of the container 2, the sensor 22, the cover 32, the spacer 26, etc., are appropriately designed within a range that achieves the effects of Embodiment 1.

For example, the shape, thickness, and diameter of the cover 32 are not particularly limited as long as the bacteria 7 existing below the cover 32 are not affected by convection occurring above the cover 32. For example, the thickness of the cover 32 may be configured to be an easy-to-handle thickness of several μm to several mm.

Further, for example, the diameter of the cover 32 does not have to be substantially the same as the inner diameter of the container 2, and a gap of a predetermined range of size may be present. In one embodiment, the gap between the outer diameter of the cover 32 and the inner diameter of the container 2 is configured to be several mm. Even with this configuration, the sedimented bacteria 7 remain between the spacers 26 as described above, so a large number of bacteria 7 do not leak above the cover 32 from the gap between the outer diameter of the cover 32 and the inner diameter of the container 2.

Further, the cover 32 does not have to be a structure that is completely in close contact with the spacer 26, and some distortion and/or bending has no effect.

As described above, the material and shape of the cover 32 are not particularly limited as long as it has a specific gravity greater than the bacterial solution 23, does not biologically and chemically affect the bacterial solution 23 and the bacteria 7, and has a diameter larger than the outer diameter of the spacer 26 and smaller than the inner diameter of the container 2, but it is desirable that it be formed of a material that is less likely to form air bubbles. In one embodiment, a hydrophilic membrane filter with a fine mesh pore size of 1 μm or less, which is often used for concentrating bacteria, is easy to handle because it instantly absorbs the bacterial solution upon contact with the bacterial solution 23 and sinks without generating air bubbles. In another embodiment, it is also preferable to use a hydrophilic membrane filter made of cellulose mixed ester with a pore size of 0.45 μm, which does not allow the bacteria 7 to pass through. As described above, by using a hydrophilic membrane filter that allows the culture medium to pass through but does not allow the bacteria 7 to pass through, it is possible to suppress the passage of the bacteria 7 through the cover 32 while also suppressing the generation of air bubbles.

It is also preferable to configure the cover 32 to include a high-molecular-weight water-absorbing polymer that absorbs the moisture of the culture medium. With this configuration, the cover 32 immediately absorbs some of the moisture in the bacterial solution 23 and covers the area above the sensor 22 without any gaps. This further restricts the movement of the bacteria 7 due to convection.

The cover 32 may also be a plate, a mesh plate, or glass formed of resin or metal that has been subjected to a surface treatment that repels bubbles. However, when using an image array sensor as shown in the embodiments described later, it is desirable that it be formed of a transparent material.

The shapes of the container 2, the sensor 22, the cover 32, the spacer 26, etc., are also not limited to the above description. For example, the container 2 may be cylindrical, and may be a cylinder with a trapezoidal cross-section, or a cylinder with a square or hexagonal cross-section. In that case, a cover 32, a sensor 22, a cover 32, a spacer 26, etc., having a shape that matches the shape of the bottom part 21 of the container 2 can be selected.

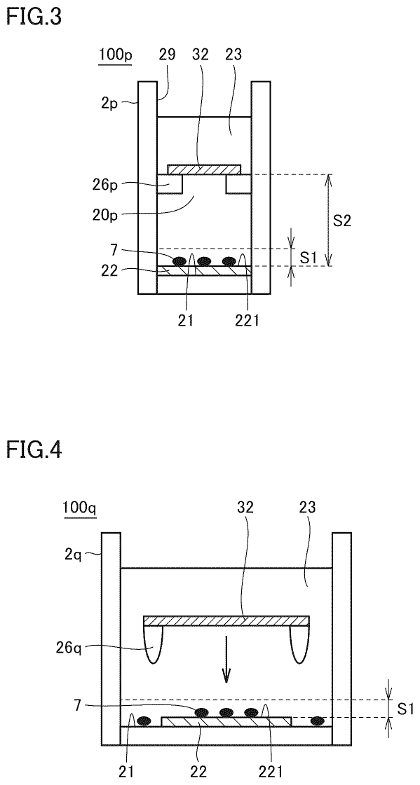

The position of the spacer 26 in Embodiment 1 is also not limited to the bottom part 21 of the container 2. FIG. 3 shows a configuration in which a spacer 26p is arranged on a side surface 29 of a container 2p. In this case, it is preferable to form the bottom part 21 of the sensor 22 to have the same shape and size as the sensor 22. As a result, all of the bacteria 7 sediment on the sensor 22 by gravity or by a centrifuge and are detected by the sensor 22. However, as shown in FIG. 1, configuring the area of the bottom part 21 to be larger than the area of the sensor 22 makes it easier to design the capacity (internal capacity) of the internal space 24 of the container 2 to be larger. As a result, in FIG. 1, a larger amount of bacteria 7 derived from the bacterial solution 23 can be used for measurement. Therefore, it is easy to secure a sufficient amount of bacteria 7 for measurement. Note that the bacterial inspection apparatuses of FIG. 3 and subsequent figures also include the analysis apparatus 4, the input device 43, and the display device 44, but their description and illustration may be omitted.

FIG. 4 shows a configuration in which a spacer 26q is arranged on the lower surface of the cover 32. However, the configuration in which the spacer 26 is arranged at the bottom part 21 as in FIG. 1 is superior in that the position of the spacer 26 does not shift from the periphery of the sensor 22 when the cover 32 is sunk.

The spacer 26 is typically fixed to the container 2 or the cover 32, but it may be configured to be detachable from the container 2 or the cover 32 as long as its position does not shift from the time the bacterial solution 23 is stored until the end of the measurement.

Further, a structure may be adopted in which the cover 32 cannot enter the detection region S of the sensor 22 by providing a slope or a step on the inner wall of the container 2, thereby using the inner wall as a spacer. FIG. 5 is a diagram showing a configuration in which a container 2r is formed in a tapered shape. The container 2r of FIG. 5 is configured such that the outer diameter of the cover 32 is larger than the outer diameter of the upper end of the detection region S. As a result, when the cover 32 is placed in the container 2, it sinks while the diameter of the inner wall of the container 2 is larger than the cover 32, and is supported where the diameter of the inner wall of the container 2 becomes equal to or smaller than the cover 32. Similarly, the upper part of the inner wall of the container may be configured to be larger than the outer diameter of the cover 32, and the lower part of the inner wall of the container may be configured to be smaller than the outer diameter of the cover 32, and the cover 32 may be supported at a step formed between the upper part and the lower part. In this case, the step is formed at a position higher than the upper end of the detection region S. As described above, in each of the configuration of providing a slope on the inner wall and the configuration of providing a step, the inner wall of the container 2 (more specifically, the part of the inner wall that supports the cover 32) functions as a spacer that supports the cover 32 in a non-contact state with the top surface 221 of the sensor 22. This can suppress the cover 32 from entering the detection region S of the sensor 22. On the other hand, the container 2 of FIG. 1 is preferable in that its shape is simpler.

Further, when the cover 32 is formed of a material that does not cause a change in the resonance frequency from the start of measurement even if it enters the detection region S on the top surface of the sensor 22, at least a part of the cover 32 may be configured to enter the detection region S. The height S2 of the spacer 26 (that is, the height of the sealed space 20) may be lower than the height S1 of the detection region S, or the spacer 26 may not be provided.

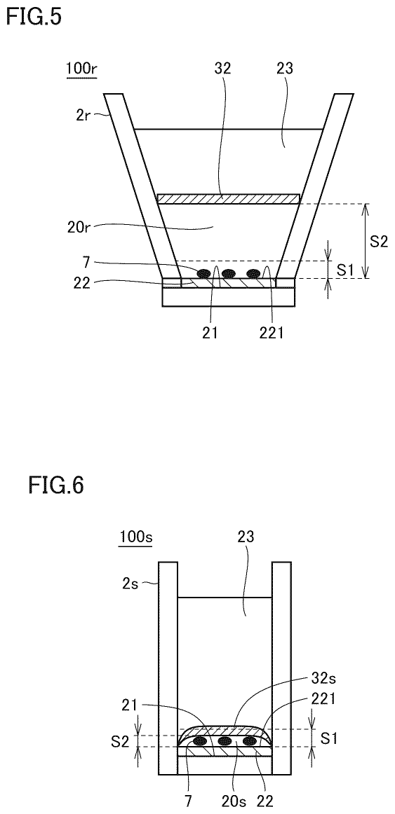

For example, by configuring the cover to be formed of a material that has a specific gravity greater than the culture medium and does not affect the detection of bacteria by the sensor 22, it is also possible to use a solid that changes its shape, as shown in FIG. 6. Since the cover 32 is a solid, it can be handled as a single piece while having flexibility, making it easier to handle than when using a liquid sealing agent. The material is a resin whose dielectric constant is lower than a predetermined value, whose specific gravity is greater than 1, and which is smaller than a predetermined value. A more specific example of the material is polypropylene terephthalate (PPT). FIG. 6 is a diagram showing a configuration using a low-rigidity cover 32s. The container unit 100s of FIG. 6 is not provided with a spacer. By dropping the cover 32s into the container 2s, the cover 32s covers the bacteria 7, thereby forming a sealed space 20s. Note that since a cover 32s with a specific gravity smaller than a predetermined value is used, even if the bacteria are pressed against the top surface 221 of the sensor 22 by the cover 32s without providing a spacer, no pressure is applied to the top surface 221 of the sensor 22. Therefore, it does not affect the detection value. As a result, similar to the configuration of FIG. 1, the state of bacteria in the bacterial solution can be accurately detected. In addition, since the cover 32s that changes its shape fits the bacteria 7 it seals, the sealed space 20s can be made smaller than the sealed space 20 of FIG. 1, and the influence of convection within the sealed space can be further reduced. On the other hand, with the configuration of FIG. 1, since the cover 32 has high rigidity, handling of the cover 32 is easy.

In the above description, an example was described in which a CMOS resonator sensor is used as the sensor 22, and the change in the dielectric constant of the bacterial solution 23 is measured as a change in the resonance frequency, as the bacteria 7 in the bacterial solution enter the detection region S of the sensor 22 and grow in the detection region S. However, the example of the sensor 22 is not limited to this, and if the shape of the bacteria 7 changes with the growth of the bacteria 7 in the bacterial solution 23, or if the refractive index or transmittance of the bacterial solution 23 changes, a CMOS image array sensor or a TFT image array sensor, which are light-receiving elements capable of measuring the change in the intensity of transmitted light, reflected light, or scattered light as a change amount, may be used.

In the embodiment, it is preferable that the bacterial inspection apparatus 1 has a structure capable of temperature control so that the temperature of the bacterial solution 23 is constant when it is affected by temperature changes. However, the invention is not limited to this, and if it is possible to monitor the temperature of the sensor 22 in real time and correct the data affected by the temperature change of the sensor 22, a structure capable of temperature control is not necessary.

The bacterial solution 23 may further contain an antibacterial agent. This makes it possible to perform a drug susceptibility test of the bacteria 7.

### Processing Procedure of Bacterial Inspection Method

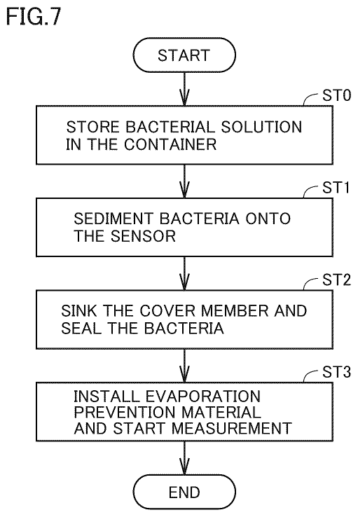

A bacterial inspection method in the bacterial inspection apparatus 1 will be described with reference to FIG. 7. FIG. 7 is a flowchart showing the processing procedure of the bacterial inspection method.

When performing a bacterial inspection in the bacterial inspection apparatus 1, first, in step ST0, a bacterial solution 23 containing bacteria 7 and a culture medium is stored in a container 2 in which a sensor 22 is provided at a bottom part 21. Specifically, for example, the bacterial solution 23 containing the bacteria 7 and the culture medium is injected into the internal space 24 of the container 2 with a pipette or the like. As a result, the bacterial solution 23 is brought into contact with the sensor 22.

Thereafter, in step ST1, the bacteria 7 are sedimented on the sensor 22. Specifically, for example, the container 2 in which the bacterial solution 23 is stored is left stationary to allow the bacteria 7 to fall onto the top surface of the sensor 22 due to gravity. Alternatively, the container 2 in which the bacterial solution 23 is stored is placed in a centrifuge. By using a centrifuge, the bacteria 7 in the container 2 can be dropped in a short time, so the time required for sedimentation of the bacteria 7 can be shortened. According to step ST1, all of the bacteria 7 in the bacterial solution 23 can be dropped onto the bottom surface of the container 2 including the top surface of the sensor 22.

In step ST2, the cover 32 is sunk into the bacterial solution 23 to seal the bacteria 7. In one embodiment, the bacteria 7 are sealed with the cover 32 and a spacer 26. Specifically, when the cover 32 is placed in the container 2, the cover 32 sinks to the top of the spacer 26 by gravity, and the space between the spacers 26 is sealed (see FIG. 1 to FIG. 5).

In another embodiment, as the cover 32s, a member formed of a material that has a specific gravity greater than the culture medium and does not affect the detection of the bacteria 7 by the sensor 22 is used. Specifically, when the cover 32s is dropped into the bacterial solution 23 in the container 2s, the cover 32s covers the bacteria 7, thereby sealing the bacteria 7 (see FIG. 6).

In step ST3, an evaporation prevention material 33 for preventing evaporation of the bacterial solution 23 is provided above the bacterial solution 23, and measurement is started. Specifically, for example, the evaporation prevention material 33 is installed on the top surface of the container 2, and the bacteria 7 existing in the detection region S of the sensor 22 are detected by the sensor 22 in the bacterial inspection apparatus 1 maintained at a temperature of 37° C. By providing the evaporation prevention material 33, it is possible to prevent the bacterial solution 23 from decreasing during measurement and affecting the growth or measurement of the bacteria 7. As the evaporation prevention material 33, a lid formed in a disk shape may be used, or a thin film may be used.

As another example of the evaporation prevention material 33, an oil or fat that prevents evaporation of the liquid in the bacterial solution 23 may be used. The liquid in the bacterial solution 23 is, for example, moisture in the culture medium. The oil or fat is, for example, an oil- or fat-based evaporation prevention seal that is generally commercially available as an evaporation prevention material. This makes it possible to suppress the evaporation of moisture in the bacterial solution 23 simply and at low cost.

By performing steps ST0 and ST2 of FIG. 7, at least a part of the bacteria 7 in the bacterial solution 23 is sealed in the sealed space 20 on the sensor 22. As a result, the state of the bacteria 7 in the bacterial solution 23 can be accurately detected.

Further, by performing step ST1 of FIG. 7, the bacteria 7 remain in the detection region S even when the cover 32 is sunk. This allows the state of the bacteria 7 to be measured stably. In addition, the height S2 of the sealed space 20 can also be configured to be higher than the height S1 of the detection region S. As a result, since the cover 32 can be placed outside the detection region S, the degree of freedom in selecting the material of the cover 32 is improved.

### Embodiment 2

Next, a configuration example will be described for a case where a bacterial inspection is performed on a plurality of containers 2 in a bacterial inspection apparatus.

When performing a bacterial inspection on a plurality of containers 2, such as a plurality of wells provided in a multi-well plate for cell culture, in a bacterial inspection apparatus, a configuration can be adopted in which one cover 32 can be inserted into each container 2. Preferably, the configuration allows one cover 32 to be inserted into each container 2 substantially simultaneously (for example, within an error of several minutes). This makes it possible to start the measurement in each container 2 substantially simultaneously.

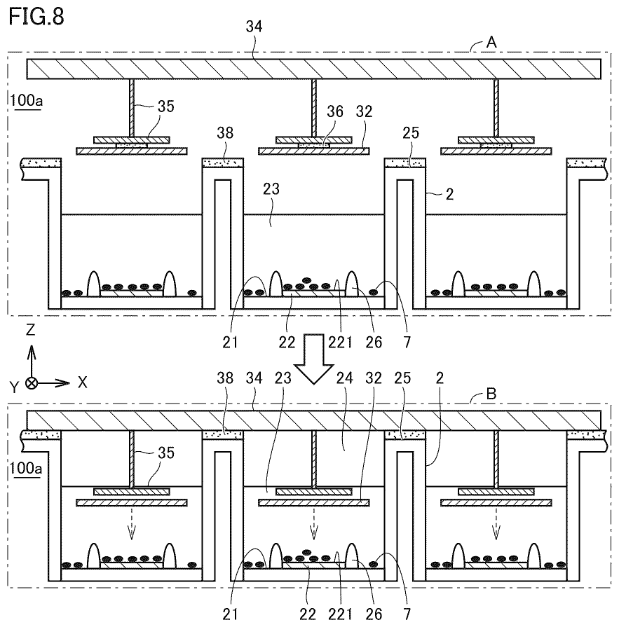

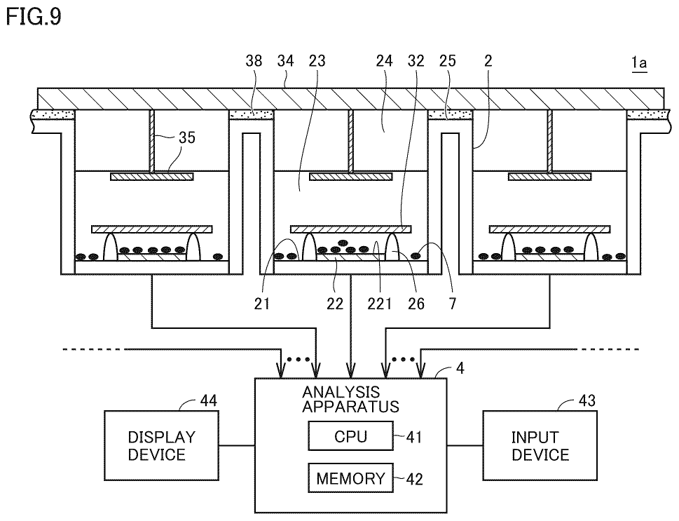

FIG. 8 and FIG. 9 are diagrams showing the configuration and operational state of a bacterial inspection apparatus 1a of Embodiment 2. FIG. 8 shows a cross-sectional view of a container unit 100a to be inspected in the bacterial inspection apparatus of the embodiment. With reference to FIG. 8 and FIG. 9, the container unit 100a includes a multi-well container 25 including a plurality of containers 2, a plurality of sensors 22 and a plurality of spacers 26 installed in the plurality of containers 2, respectively, and a plurality of covers 32 corresponding to the plurality of containers 2.

As in Embodiment 2, when performing a bacterial inspection on a plurality of containers 2, if the plurality of containers 2 are integrated, handling of the plurality of containers 2 is easy. In the example of FIG. 8, the container 2 corresponds to each of a plurality of wells on a multi-well container 25. Examples of the multi-well container 25 are 6-, 12-, 24-, 48-, and 96-multi-well plates. The multi-well container 25 may be formed with more than 96 wells.

Preferably, the plurality of covers 32 are also configured such that their mutual positional relationship on the XY plane is fixed using one or more members. This makes it easy to correctly position each of the plurality of covers 32 above the sensor 22 of the corresponding container 2.

For example, the plurality of covers 32 can be connected and their mutual positional relationship on the XY plane can be fixed by connecting them to each other at least temporarily via one or more members. In one embodiment, each cover 32 is temporarily connected to each other via a multi-well cover 34, which is a cover of the multi-well container 25, whereby they are connected, and their mutual positional relationship in the XY direction is fixed. The multi-well cover 34 corresponds to one embodiment of a “connecting member.”

Preferably, a member for bringing the multi-well container 25 and the multi-well cover 34 into close contact is provided at a portion where the multi-well container 25 and the multi-well cover 34 are in contact. In the example of FIG. 8, a double-sided adhesive sheet 38 is provided at the upper end of the container 2. The double-sided adhesive sheet 38 is typically a sheet that is so thin as to be negligible with respect to the depth of the container 2. With this configuration, when the multi-well cover 34 is placed on the multi-well container 25, both are brought into close contact via the double-sided adhesive sheet 38. This can prevent the multi-well container 25 and the multi-well cover 34 from being unintentionally displaced. In particular, by bringing the space between each container 2 and the adjacent container 2 into close contact, it is possible to prevent the bacterial solution 23 from leaking between each container 2 and the adjacent container 2. This reduces the risk of contamination and cross-contamination.

The container unit 100a further includes one or more support members 35 connected to the upper part of the cover 32. In the example of FIG. 8 and FIG. 9, the cover 32 is connected to the support member 35 by a water-soluble adhesive 36. The upper end of the support member 35 is connected to the multi-well cover 34. As described above, each of the plurality of covers 32 is connected to the multi-well cover 34 via a plurality of adhesives 36 and a plurality of support members 35. As a result, the plurality of support members 35 are directly connected. As a result, the plurality of covers 32 are indirectly connected. The support member 35 corresponds to one embodiment of a “support member” and also corresponds to one embodiment of a “positioning member.” A plurality of support members 35 (described later) are connected.

In one embodiment, the support member 35 is a member in which a disk for adhering the adhesive 36 and a rod-shaped member connecting the disk and the multi-well cover 34 are joined.

The position of the support member 35 with respect to the multi-well cover 34 is arranged such that each cover 32 is positioned above each sensor 22 of each container 2. The maximum dimension of the support member 35 in the XY direction is designed to be smaller than the inner diameter of each container 2. This allows the support member 35 to which the cover 32 is adhered to be inserted into the container 2, as shown in the first state A of FIG. 8.

Further, the length of the support member 35 in the Z direction is designed such that after the bacterial solution 23 is stored in the container 2 and the multi-well cover 34 is installed on the multi-well container 25, the adhesive 36 is positioned lower than the liquid level of the bacterial solution 23 and higher than the upper end of the spacer 26. For example, the length of the support member 35 in the Z direction is configured to be longer than the value obtained by subtracting the height of the liquid level of the bacterial solution 23 from the depth of the container 2, and shorter than the value obtained by subtracting the height of the spacer 26 with respect to the bottom part 21 from the depth of the container 2. With this configuration, as shown in the second state B of FIG. 8, when the multi-well cover 34 is installed on the multi-well container 25, the adhesive 36 is positioned at a position higher than the upper end of the spacer 26 in the bacterial solution 23. By dissolving the adhesive 36, the cover 32 falls onto the upper end of the spacer 26, and the state shown in FIG. 9 is reached.

In the bacterial inspection apparatus 1a, when a bacterial inspection is performed on a plurality of containers 2, after one cover 32 is inserted as shown in FIG. 2 in each container 2, detection of the bacteria 7 in the bacterial solution 23 is performed by the corresponding sensor 22. Whether the insertion of the cover 32 into each container 2 and the detection of the bacteria 7 by the sensor 22 in each container 2 are executed simultaneously or in a predetermined order is set as appropriate.

Detection signals from the plurality of sensors 22 are input to the analysis apparatus 4. In the analysis apparatus 4, a program for analyzing the bacterial inspection of each container 2 in response to the detection signals input from the plurality of sensors 22 is stored in the ROM, and by executing the program, the bacterial inspection of each container 2 is performed on the bacterial solution 23 stored in each of the plurality of containers 2.

As described above, according to the configuration in which a plurality of covers are provided for a plurality of sensors 22 housed in a plurality of containers 2, it is possible to perform a bacterial inspection on a plurality of containers 2.

Further, by providing one or more positioning members (in the example of FIG. 8 and FIG. 9, the support members 35) for determining the position of the cover above the cover 32a, the position of the cover 32 on the XY plane can be controlled. This makes it easier to control the position of the cover 32 than when dropping the cover 32 into the bacterial solution 23 in the container unit 100 of FIG. 1. Therefore, the cover can be more reliably placed at a desired position.

Furthermore, by operating the support member 35 connected to the upper part of the cover 32, it becomes even easier to control the position of the cover 32.

In addition, by also including a connecting member (in the example of FIG. 8 and FIG. 9, the multi-well cover 34) that connects the plurality of support members 35 in addition to the plurality of support members 35, the position of the cover 32 can be positioned at once for a plurality of containers 2. In addition, it also becomes easy to simultaneously insert a plurality of covers 32 into a plurality of containers 2, respectively. This also makes it easy to simultaneously start the culture and measurement of the bacteria 7 in a plurality of containers 2.

Further, by using the multi-well cover 34 as the connecting member, it can also serve as a lid for the upper part of each container 2. This can prevent contamination, cross-contamination, a decrease in the bacterial solution 23 due to evaporation, and the like.

The sizes, shapes, and materials of the positioning member, the support member, and the connecting member are not limited to the above examples and may be appropriately designed. For example, as the positioning member, it is also possible to position the cover 32 by making a hole of the size of the cover 32 at a position corresponding to the plurality of containers 2 of the multi-well cover 34 and dropping the cover 32 through the hole.

### Growth Determination of Bacteria in Bacterial Inspection Apparatus

By detecting the bacteria 7 in the bacterial solution 23 by the bacterial inspection apparatus 1 described in the above embodiments, the growth status of the bacteria can be monitored in real time. An example thereof will be shown below.

### Embodiment 3

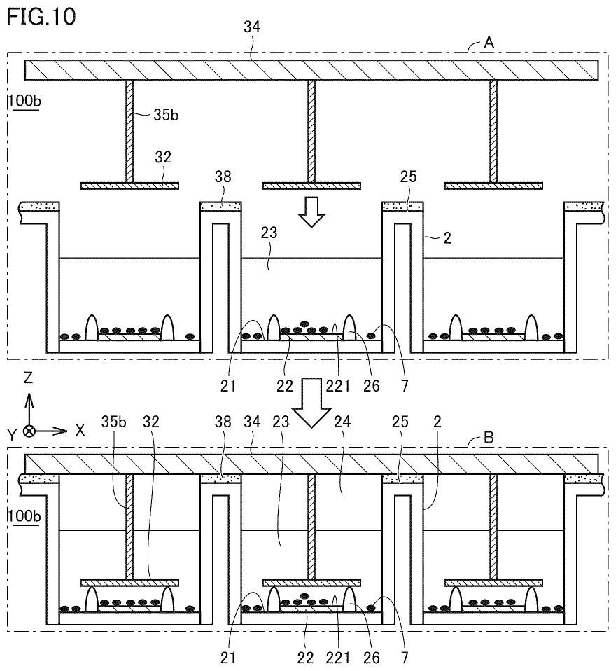

FIG. 10 is a diagram showing the configuration and operational state of a bacterial inspection apparatus 1b according to Embodiment 3.

The difference between the container unit 100b of the bacterial inspection apparatus 1b and the container unit 100a of the bacterial inspection apparatus 1a is that the support member 35 is replaced by a support member 35b, and the support member 35b is joined to the cover 32 without interposing a water-soluble adhesive 36. However, the support member 35b may be adhered to the cover 32 by an adhesive having a property of not dissolving in water.

In one embodiment, the support member 35b is, for example, a rod-shaped member connecting the cover 32 and the multi-well cover 34. The support member 35b corresponds to one embodiment of a “support member” and also corresponds to one embodiment of a “positioning member.”

In Embodiment 3, the length of the support member 35b is configured to be equal to the length between the upper end of the container 2 and the upper end of the sealed space 20. As a result, when the multi-well cover 34 is placed on the multi-well container 25, a plurality of sealed spaces 20 can be easily formed.

In the example using the spacer 26 as in FIG. 10, the length of the support member 35b is designed such that the cover 32 reaches the upper end of the spacer 26 when the multi-well cover 34 is installed on the multi-well container 25 after the bacterial solution 23 is stored in the container 2. For example, the length of the support member 35b in the Z direction is configured to be equal to the value obtained by subtracting the height of the spacer 26 with respect to the bottom part 21 from the depth of the container 2. More accurately, the sum of the length of the support member 35b and the thickness of the cover 32 is configured to be the length between the upper end of each container 2 and the upper end of the spacer 26. With this configuration, as shown in the second state B of FIG. 10, by simply placing the multi-well cover 34 on the multi-well container 25, the cover 32 can seal the space between the spacers 26, and a sealed space 20b can be formed.

With this configuration, since a plurality of covers 32 can be integrally configured with the multi-well cover 34, handling of the plurality of covers 32 is easy. In addition, the time for waiting for the water-soluble adhesive 36 to dissolve can be shortened. Further, the sealed space 20b can be formed in each container 2 more accurately and simultaneously (for example, within an error of 1 second) than when using the water-soluble adhesive 36. As described above, according to Embodiment 3, a bacterial inspection can be performed with a simpler structure and more simply than in Embodiment 2.

Further, by configuring the length of the support member 35b to be equal to the length between the upper end of the container 2 and the upper end of the sealed space 20 (more accurately, by configuring the sum of the length of the support member 35b and the thickness of the cover 32 to be the length between the upper end of each container 2 and the upper end of the spacer 26), even in a cylindrical container 2, a sealed space can be formed without providing a spacer, provided that the size of the sensor 22 and the size of the bottom part 21 are configured to be equal as in FIG. 3 and the diameter of the cover 32 is configured to be equal to the diameter of the container 2.

### Embodiment 4

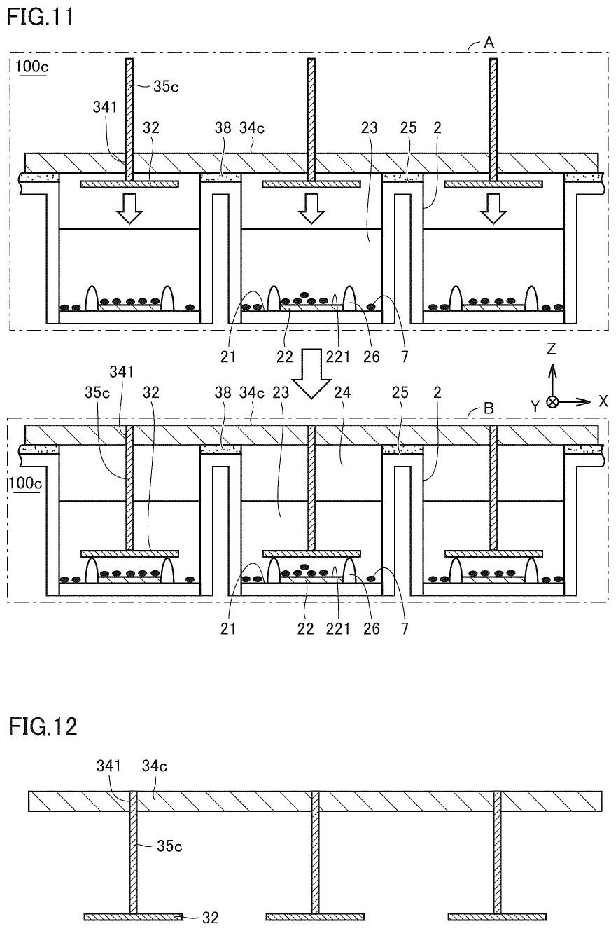

FIG. 11 is a diagram showing the configuration and operational state of a bacterial inspection apparatus of Embodiment 4.

In the container unit 100c of the bacterial inspection apparatus 1c, the multi-well cover 34 and the support member 35b of the container unit 100b of the bacterial inspection apparatus 1b are replaced by a multi-well cover 34c and a support member 35c. The support member 35c corresponds to one embodiment of a “support member” and also corresponds to one embodiment of a “positioning member.”

FIG. 12 is a diagram showing the structure of the multi-well cover 34c, the support member 35c, and the cover 32 of Embodiment 4.

The multi-well cover 34c is provided with a hole 341 for passing the support member 35c up and down. This allows the distance between the multi-well cover 34c and each cover 32 to be changed. The multi-well cover 34c corresponds to one embodiment of a “connecting member” and also corresponds to one embodiment of a “lid member.”

The length of the support member 35c is configured to be longer than the length between the upper end of the container 2 and the upper end of the sealed space 20. This allows the sealed space 20 to be formed by lowering the cover 32 joined to the lower end of the support member 35c.

In one embodiment, the length of the support member 35c is configured to be the length obtained by adding the thickness of the multi-well cover 34c to the length between the upper end of the container 2 and the upper end of the sealed space 20. With this configuration, in a state where the support member 35c has moved to the lowermost position and the sealed space 20 is formed, the upper end of the support member 35c does not protrude from the top surface of the multi-well cover 34c, so handling of the container unit 100c becomes easy.

In the example using the spacer 26 as in FIG. 11, the length of the support member 35c is designed to allow the cover 32 to be lowered to the upper end of the spacer 26 after the bacterial solution 23 is stored in the container 2 and the multi-well cover 34c is installed on the multi-well container 25. For example, the length of the support member 35c is configured to be greater than the value obtained by subtracting the height of the spacer 26 with respect to the bottom part 21 from the depth of the container 2. With this configuration, as shown in the second state B of FIG. 11, after installing the multi-well cover 34c on the multi-well container 25, by moving the support member 35c downward along the hole 341, the space between the spacers 26 can be sealed by the cover 32, and a sealed space 20c can be formed.

In FIG. 11, a first state A shows a state in which, after injecting the bacterial solution 23 into the container 2 with a pipette or the like, the multi-well cover 34c is placed on the multi-well container 25, and then the bacteria 7 are sedimented by a centrifuge. In this state, the cover 32 is placed at a position where it does not reach the liquid level of the bacterial solution 23. In other words, the distance between the lower surface of the multi-well cover 34c and the lower surface of each cover 32 is adjusted to be smaller than the value obtained by subtracting the height of the liquid level of the bacterial solution 23 from the depth of the container 2.

In one embodiment, the multi-well cover 34 is designed such that the support member 35c does not unintentionally move up and down with respect to the hole 341 while being subjected to a centrifuge or the like. This prevents the cover 32 from unintentionally dropping into the bacterial solution 23 while being subjected to a centrifuge or the like.

A second state B shows a state in which the sealed space 20c is formed by moving the support member 35c downward through the hole 341 to bring the cover 32 to the upper end of the spacer 26. Specifically, by pushing the upper end of the support member 35c protruding from the top surface of the multi-well cover 34 downward, the cover 32 connected to the lower end of the support member 35c is pushed down.

As described above, according to Embodiment 4, since the water-soluble adhesive 36 is not used as in Embodiment 2, the structure and handling of the container unit 100c are simple. In addition, compared to Embodiment 2 and Embodiment 3, after storing the bacterial solution and covering with the multi-well cover 34, it is possible to keep each container 2 sealed with the multi-well cover 34 until the measurement is completed after sedimenting the bacteria by a centrifuge or gravity.

The positioning member and the support member of Embodiments 2 to 4 can also be applied to a case where a bacterial inspection is performed on a single container 2, not a plurality.

It is also possible to use a plurality of positioning members and a plurality of support members for a plurality of containers without connecting them to each other directly or indirectly. However, when using a plurality of containers, it is preferable to adopt the configuration of FIG. 8 to FIG. 11 in which each of the plurality of positioning members and the plurality of support members are used by being connected to each other directly or indirectly, because the structure of the container unit can be simplified and the operation is easy.

### Comparison With Conventional Bacterial Inspection Method

In a conventional bacterial inspection method using a conventional bacterial inspection apparatus as shown in Non-Patent Literature 1, in a state where the depth of a bacterial solution stored in a container such as a well exceeds the detection region of a sensor provided on the bottom surface of the container, the sensor cannot detect bacteria in the bacterial solution outside the detection region. This made it difficult for the sensor to accurately detect the state of bacteria in the bacterial solution.

Furthermore, there was a problem that the bacterial solution in the container convected due to the heat generated from the sensor and a slight temperature difference between the upper and lower parts of the container, causing the bacteria to not remain uniformly in the sensor detection region but to become unevenly distributed, or to move due to convection, making accurate measurement impossible.

In addition, for slow-growing bacteria, a long measurement time is required, and there was a problem that it was not possible to determine whether the dielectric constant of the bacterial solution in the detection region changed because the bacteria that fell by gravity gradually fell into the sensor detection region, or whether the dielectric constant changed because the bacteria grew. To solve this problem, it was also necessary to wait for the bacteria to fall before starting the measurement.

In the bacterial inspection apparatus according to each of the above embodiments, by sealing the area above the sensor with a cover and creating a small sealed space, it is possible to prevent bacteria from moving into and out of the detection region of the sensor due to convection or falling by gravity. In particular, by separating the cover and the sensor, or by using a cover with a low specific gravity, it is possible to prevent pressure from being applied to the top surface of the sensor and affecting the measurement. This allows the sensor to detect bacteria in the bacterial solution accurately, stably, and with good reproducibility. In addition, since the bacteria can be dropped in a short time by a centrifuge instead of waiting for a long time for the bacteria to fall by gravity, the measurement throughput can be improved.

### EXAMPLE

In this example, BCG, which is biologically similar to Mycobacterium tuberculosis and has a doubling time of 15 hours or more, was used as the bacteria 7. A CMOS resonator sensor was used as the sensor 22.

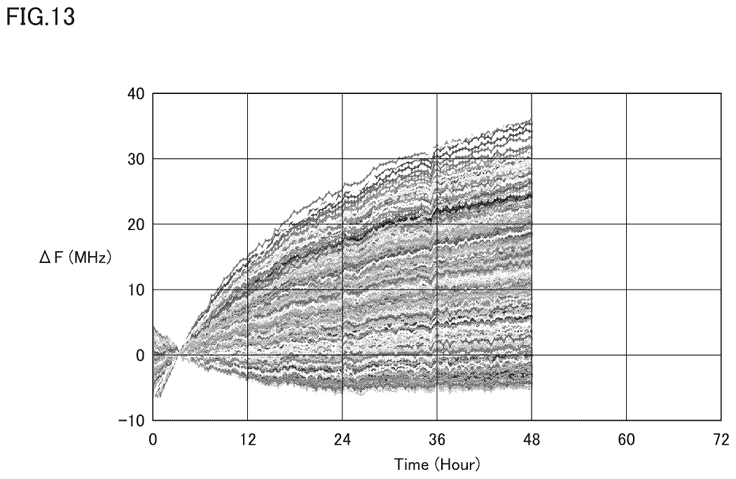

FIG. 13 is a diagram showing the change over time of the resonance frequency when a bacterial solution 23 containing bacteria 7 and a culture medium was stored in a container 2 and measurement was started immediately (without waiting for the bacteria 7 to fall by gravity) without using a cover 32. The horizontal axis of FIG. 13 indicates time, and the vertical axis indicates the amount of change in resonance frequency (ΔF). FIG. 13 is a graph of time-series data obtained by acquiring the amount of change in resonance frequency (ΔF) in about 1000 elements of the array sensor constituting the sensor 22 over 48 hours. Since the CMOS resonator sensor reacts very sensitively to temperature changes, measurement cannot be performed accurately for several hours after placing the container 2 at a room temperature of about 25° C. in the bacterial inspection apparatus 1 and starting the measurement until the temperature of the sensor 22 stabilizes at the set temperature of 37° C. Therefore, in this example, the resonance frequency of each element 4 hours after the start of measurement, when the temperature of the sensor 22 stabilized, was used as a reference frequency, and the difference between the resonance frequency obtained at each time point and the reference frequency was defined as the amount of change in resonance frequency (ΔF). Therefore, in the graph of FIG. 13, ΔF at 4 hours is zero.

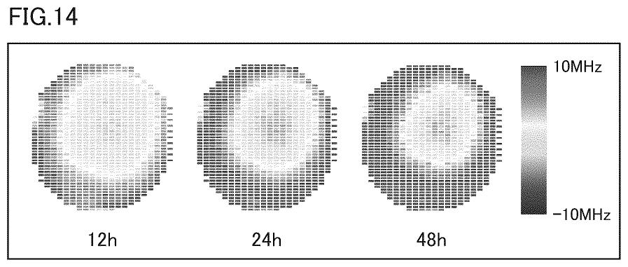

FIG. 14 is a diagram in which the ΔF at 12 hours, 24 hours, and 48 hours after the start of measurement shown in the data of FIG. 13 is converted to grayscale from a two-dimensional map image displayed in a color scale according to the arrangement of each element. When the dielectric constant does not change, the amount of change in resonance frequency (ΔF) is almost 0, which is represented by yellowish green (white in grayscale) in the two-dimensional map image. As the dielectric constant decreases with the growth of the bacteria 7, the resonance frequency increases, and ΔF becomes larger, so it is represented by a more reddish color (a more blackish color in grayscale) in the two-dimensional map image.

Referring to the change over time of ΔF for each element in FIG. 13, it showed a convex logarithmic function-like change, and elements ranging from those that increased rapidly to small elements that gradually shifted to the negative side were observed. This shows that there is a large difference in how the resonance frequency changes (for example, the sign of ΔF, the absolute value of ΔF, the amount of change in ΔF, etc.) depending on the element. Referring to FIG. 14, the central part of the sensor 22 showed a yellowish green color, and the peripheral part showed a red color. This shows that the resonance frequency hardly increased in the central part of the sensor 22, and the resonance frequency greatly increased from the central part to the peripheral part of the sensor 22.

The doubling time of Mycobacterium tuberculosis is said to take 15 hours or more, but referring to FIG. 13, there were a plurality of elements whose frequency increased rapidly even after 4 hours from the start of measurement when the temperature had already stabilized. As a result of repeated experiments and studies on this phenomenon, it was found that this was due to the effect of the falling of BCG by gravity over 20 hours or more, and the bacterial solution 23 convecting in the container 2 due to a minute temperature gradient in the container 2 and the heat received from the sensor 22, causing the bacteria 7 to move to a local place in the container 2 (in this example, the peripheral part of the sensor 22) and become unevenly distributed.

Instead of waiting for the bacteria 7 to fall by gravity in step ST2, if the bacterial solution 23 is centrifuged to sediment all the bacteria 7 contained in the bacterial solution 23 in the detection region S of the sensor 22 in a short time, it is thought that the sensor 22 will be able to detect the bacteria 7 in the bacterial solution 23 more quickly and reliably without being affected by the falling by gravity. In addition, since the concentration of the bacteria 7 contained in the bacterial solution 23 in the detection region S of the sensor 22 can be increased quickly, it is possible to detect the growth state from the initial stage of culture even with a small number of bacteria, and it is thought that the time required for the culture test can be shortened. However, the influence of convection cannot be avoided.

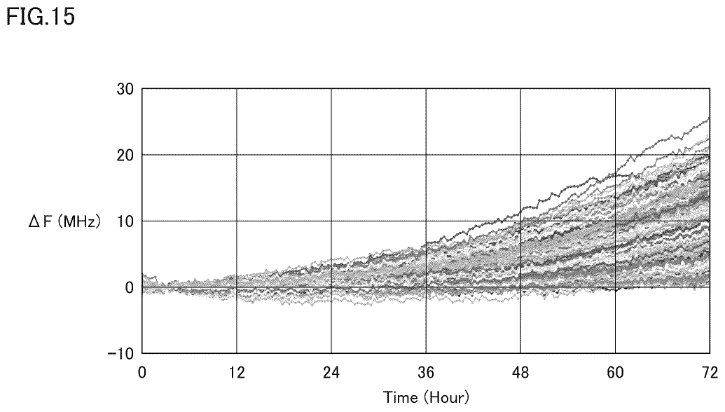

FIG. 15 is a diagram showing the change over time of the resonance frequency when a bacterial solution 23 with BCG as the bacteria 7, prepared in the same manner as in FIG. 13, was centrifuged in advance, and a cover 32 was dropped as shown in the embodiment described above, and measurement was started. The horizontal axis of FIG. 15 indicates time, and the vertical axis indicates ΔF. FIG. 15 is a graph of time-series data obtained by acquiring the amount of change in resonance frequency (ΔF) in about 1000 elements of the array sensor constituting the sensor 22 over 72 hours. Similar to FIG. 13, in FIG. 15, the resonance frequency of each element 4 hours after the start of measurement was used as a reference frequency, and the difference between the resonance frequency obtained at each time point and the reference frequency was defined as the amount of change in resonance frequency (ΔF). Therefore, in the graph of FIG. 15, the data value at 4 hours is zero.

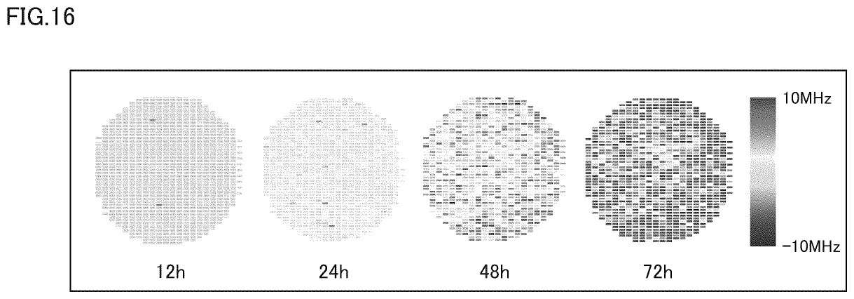

FIG. 16 is a diagram in which the ΔF at 12 hours, 24 hours, 48 hours, and 72 hours after the start of measurement shown in the measurement data of FIG. 15 is converted to grayscale from a two-dimensional map image displayed in a color scale according to the arrangement of each element. When bacteria are present and grow in the detection region S of each element, or when new bacteria enter the detection region S due to convection, or in both cases, the bacteria in the detection region S increase, the resonance frequency increases, and ΔF also becomes larger. Therefore, the increase in the bacteria is represented by a red color in the two-dimensional map image.

Referring to the change over time of ΔF for each element in FIG. 15, although the amount of change in resonance frequency (ΔF) differed depending on the element, it showed a concave exponential growth curve similar to the general growth curve of bacteria. In addition, the amount of change in resonance frequency (ΔF) depended on the number of bacteria 7 (more specifically, the bacterial density) existing in the detection region S of each element, and the larger the number of the bacteria 7, the larger the amount of change in resonance frequency (ΔF). Referring to FIG. 16, although most of the elements were yellowish green at 12 hours, a tendency was seen for the entire area to change to yellow and red as time passed to 24 hours, 48 hours, and 72 hours. In FIG. 16, unlike FIG. 14, uneven distribution of colors was not seen, and the change in color occurred uniformly without bias between the central part and the peripheral part. Therefore, from FIG. 16, it can be seen that the bacteria 7 sedimented almost uniformly in the detection region S of the sensor 22, and then grew while remaining at their original positions without moving and becoming unevenly distributed due to convection.

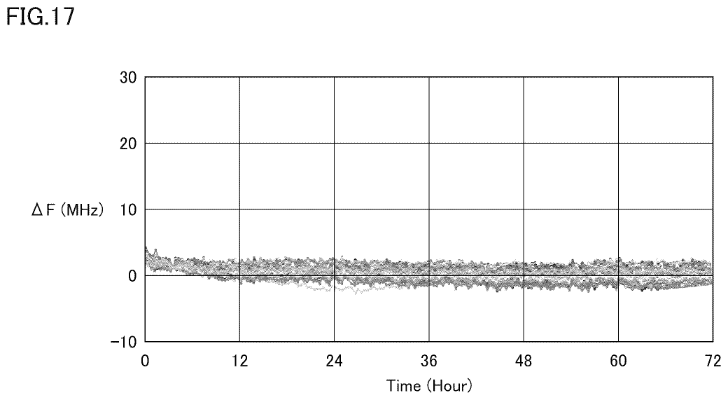

FIG. 17 is a graph of time-series data of ΔF of a culture medium not containing bacteria, which was obtained over 72 hours in the same manner as in FIG. 15 as a control experiment.

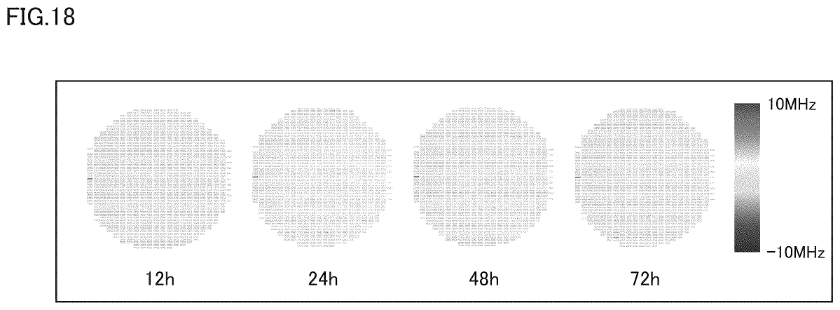

FIG. 18 is a diagram in which the ΔF at 12 hours, 24 hours, 48 hours, and 72 hours after the start of measurement shown in the measurement data of FIG. 17 is converted to grayscale from a two-dimensional map image displayed in a color scale according to the arrangement of each element. The two-dimensional maps at 12 hours, 24 hours, 48 hours, and 72 hours in FIG. 18 showed a yellowish green color overall and did not change over time.

From FIG. 17 and FIG. 18, it was found that when the detection region S of the sensor 22 does not contain bacteria, ΔF fluctuates somewhat over time due to the influence of noise components peculiar to the sensor, system noise of the bacterial inspection apparatus 1, etc., but is almost constant. More specifically, from 24 hours to 72 hours after culture, the S.D. of ΔF of all elements showed a small value of about 0.35 MHz.

### First Method of Determining Bacterial Growth in Bacterial Inspection Apparatus

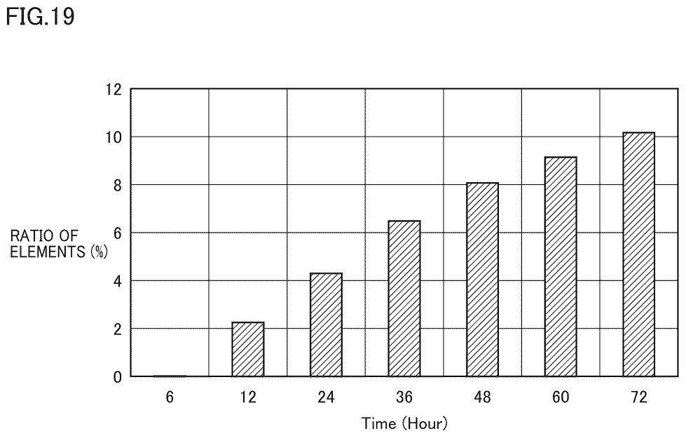

FIG. 19 is a graph showing the ratio of the number of elements with ΔF exceeding 1 MHz to the total number of elements over time, by setting a threshold of ΔF=1 MHz for the data obtained in FIG. 15. If a time-series increase is recognized in the graph of FIG. 19, it can be determined that the bacteria 7 have increased. In addition, if the threshold is not exceeded in all elements, or if the ratio of the number of elements does not increase, it can be determined that the bacteria have not grown, or that the bacteria could not be detected because the bacterial concentration was low. Referring to FIG. 19, since the ratio of the number of elements with a change in resonance frequency (ΔF) exceeding 1 MHz to the total number of elements is increasing, it can be said that an increase in the bacteria 7 was detected in the bacterial solution 23 corresponding to the data of FIG. 15.

### Second Method of Determining Bacterial Growth in Bacterial Inspection Apparatus

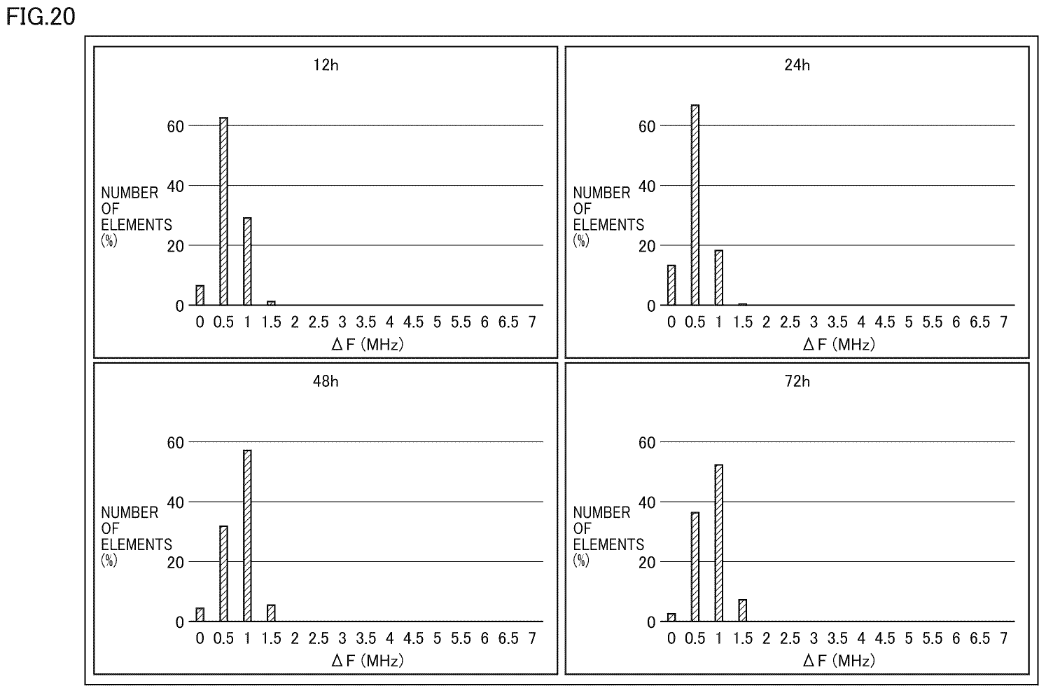

FIG. 20 is a histogram of the change in resonance frequency of all elements at 12 hours, 24 hours, 48 hours, and 72 hours from the data of the culture medium not containing the bacteria 7 of FIG. 17. There is almost no difference between 12 hours and 72 hours.

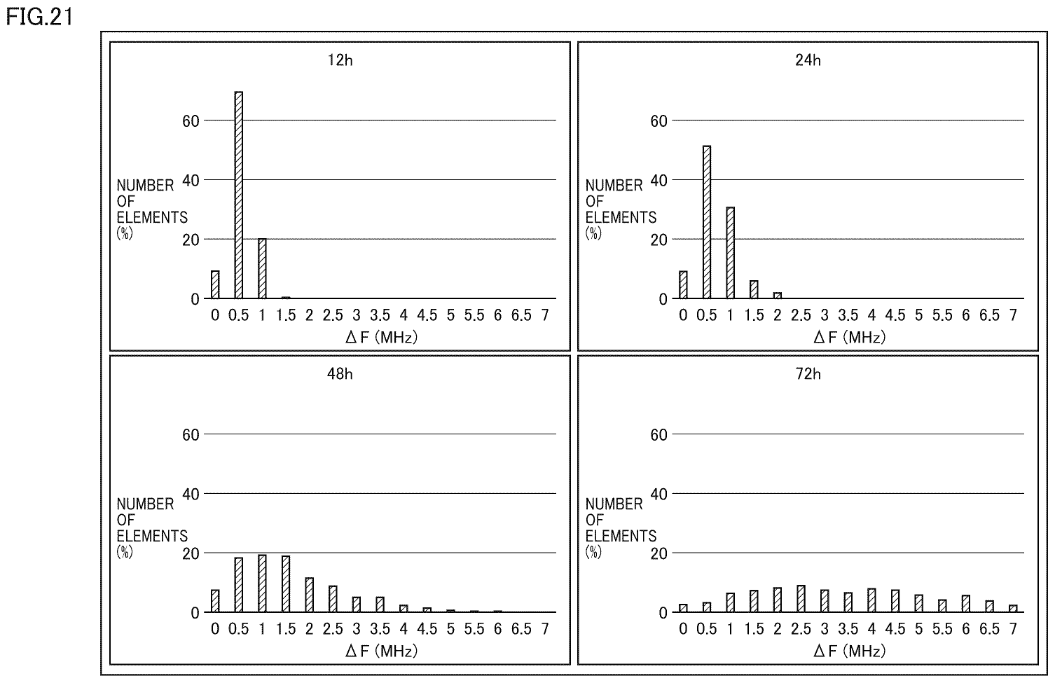

FIG. 21 is a histogram of the change in resonance frequency of all elements at 12 hours, 24 hours, 48 hours, and 72 hours from the data of the bacterial solution 23 containing the bacteria 7 of FIG. 15. It can be seen that the frequency distribution of the histogram changes over time, and the resonance frequency increases in many elements. Therefore, it can be seen that the bacteria are increasing in the detection region S of many elements.

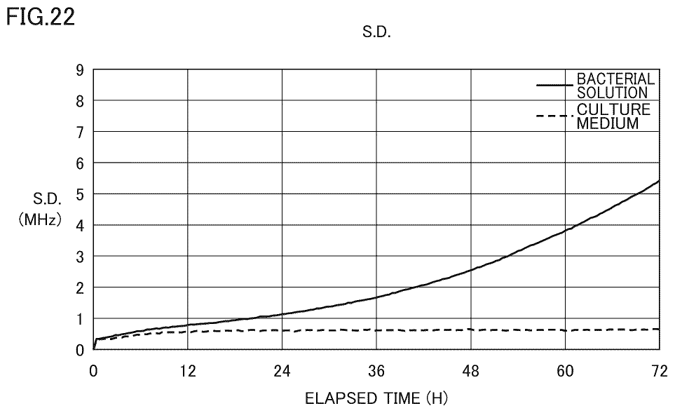

FIG. 22 is a graph of the change over time of the S.D. of ΔF of the bacterial solution 23 of FIG. 15 and the change over time of the S.D. of the resonance frequency of the culture medium not containing the bacteria 7 of FIG. 17. The S.D. of ΔF is an index of the dispersion (variation) of ΔF, and as the bacteria grow, the dispersion of ΔF expands, so the S.D. of ΔF also increases. The S.D. of ΔF of the culture medium did not change over time, but the S.D. of ΔF of the bacterial solution 23 increased over time.

According to FIG. 20 to FIG. 22, when the bacterial solution 23 was cultured and measured using the bacterial inspection apparatus according to the embodiment, it can be seen that the bacteria were distributed on many elements and grew on each element.

As described above, it is possible to determine the growth of the bacteria 7 based on the frequency distribution of the histogram and/or the S.D. More specifically, it can be seen that the frequency distribution of the histogram of the bacterial solution 23 changes to include more components with a large ΔF, and the S.D. of ΔF of the bacterial solution 23 increases, so that the bacteria 7 are uniformly dispersed and sedimented in the detection region S of the sensor 22 by the centrifuge and are growing.

From the above results, it can be said that when measuring the growth state of bacteria, the bacterial inspection method including the step of sealing bacteria in the sealed space 20 above the sensor using the cover 32, as shown in the embodiment above, and the bacterial inspection apparatus having a structure for sealing bacteria in the sealed space 20 above the sensor using the cover 32, have a remarkable effect and are clearly effective.

### Aspects

As described above, the present embodiment includes the following disclosures. In other words, it is understood by those skilled in the art that the plurality of exemplary embodiments described above are specific examples of the following aspects.

### Configuration 1

A bacterial inspection apparatus comprising: one or more containers for storing a bacterial solution containing bacteria and a culture medium; a sensor provided at the bottom of each of the one or more containers and having a bacteria detection region above its top surface; a cover for sealing the bacteria; and a spacer for supporting the cover in a non-contact state with the top surface of the sensor.

According to such a configuration, the cover covers the area above the sensor, so that the movement of bacteria is restricted in the sealed space below the cover. In addition, since the cover and the top surface of the sensor are separated, no pressure is applied to the top surface of the sensor, so the frequency change of the sensor due to the growth of the bacteria can be accurately measured. As described above, the state of bacteria in the bacterial solution can be accurately detected.

### Configuration 2

The bacterial inspection apparatus according to Configuration 1, wherein the diameter of the cover is larger than the diameter of the opening formed by the spacer.

According to such a configuration, a sealed space can be formed with only two simple members, a cover and a spacer.

### Configuration 3

The bacterial inspection apparatus according to Configuration 1 or Configuration 2, wherein the spacer is arranged at the bottom of the container.

According to such a configuration, the position of the spacer does not shift from the periphery of the sensor when the cover is sunk.

### Configuration 4

The bacterial inspection apparatus according to any one of Configurations 1 to 3, further comprising an evaporation prevention material provided above the bacterial solution for preventing evaporation of the bacterial solution.

According to such a configuration, it is possible to prevent the bacterial solution from decreasing during measurement and affecting the growth or measurement of the bacteria.

### Configuration 5

The bacterial inspection apparatus according to Configuration 4, wherein the evaporation prevention material includes an oil or fat for preventing evaporation of moisture in the bacterial solution.

According to such a configuration, an oil- or fat-based evaporation prevention seal that is generally commercially available as an evaporation prevention material can be used as the evaporation prevention material, and it is possible to prevent the evaporation of moisture in the bacterial solution simply and at low cost.

### Configuration 6

The bacterial inspection apparatus according to any one of Configurations 1 to 5, further comprising one or more positioning members above the cover for determining the position of the cover.

According to such a configuration, the cover can be more reliably placed at a desired position.

### Configuration 7

The bacterial inspection apparatus according to any one of Configurations 1 to 6, wherein the bacterial inspection apparatus includes one or more support members connected to the upper part of the cover.

According to such a configuration, it becomes even easier to control the position of the cover.

### Configuration 8

The bacterial inspection apparatus according to Configuration 7, wherein the one or more containers are a plurality of containers, the plurality of containers respectively correspond to a plurality of wells on a multi-well plate, the one or more support members are a plurality of support members, and the bacterial inspection apparatus further comprises a connecting member for connecting the plurality of support members.

According to such a configuration, the position of the cover can be positioned at once for a plurality of containers. In addition, it also becomes easy to simultaneously insert a plurality of covers into a plurality of containers, respectively. This also makes it easy to simultaneously start the culture and measurement of the bacteria in a plurality of containers.

### Configuration 9

The bacterial inspection apparatus according to Configuration 8, wherein the length of each of the plurality of support members is configured such that the sum of the length of each of the plurality of support members and the thickness of the cover is the length between the upper end of each of the plurality of containers and the upper end of the spacer.

According to such a configuration, when the support member is fixed to the lower surface of the lid member, a plurality of sealed spaces can be easily formed by simply placing the lid member on the container.

### Configuration 10

The bacterial inspection apparatus according to any one of Configurations 7 to 9, further comprising a lid member provided with a hole for passing each of the one or more support members up and down, respectively.

According to such a configuration, after storing the bacterial solution and covering with the lid member, it is possible to keep each container sealed with the lid member until the measurement is completed after sedimenting the bacteria by a centrifuge or gravity.

### Configuration 11

The bacterial inspection apparatus according to any one of Configurations 1 to 10, wherein the cover seals the bacteria in a state of being sedimented on the sensor.

According to such a configuration, a larger number of bacteria can be targeted for measurement, and thus stable measurement can be performed. In addition, the sealed space can be configured to be larger than the detection region.

### Configuration 12

The bacterial inspection apparatus according to Configuration 11, wherein the cover seals the bacteria in a state of being sedimented on the sensor by a centrifuge.

According to such a configuration, since the bacteria can be sedimented in a short time, the measurement throughput can be further improved.

### Configuration 13

The bacterial inspection apparatus according to any one of Configurations 1 to 12, wherein the cover includes a hydrophilic membrane filter that allows the culture medium in the bacterial solution to pass through but does not allow the bacteria to pass through.

According to such a configuration, when the cover is placed in the container, the cover can instantly absorb the bacterial solution and sink without generating air bubbles.

### Configuration 14

The bacterial inspection apparatus according to any one of Configurations 1 to 13, wherein the cover is formed of a material that has a specific gravity greater than the culture medium and does not affect the components of the culture medium.

According to such a configuration, for example, since the cover can be formed of a resin, metal, glass, or the like that satisfies the above two conditions, the cover sinks to the upper part of the spacer without distortion or bending, and does not shift due to shaking when moving the container.

### Configuration 15

The bacterial inspection apparatus according to any one of Configurations 1 to 14, wherein the cover includes a high-molecular-weight water-absorbing polymer that absorbs the moisture of the culture medium.

According to such a configuration, the cover immediately absorbs some of the moisture in the bacterial solution and covers the area above the sensor without any gaps. This further restricts the movement of the bacteria due to convection.

### Configuration 16

A bacterial inspection apparatus, comprising: a container for storing a bacterial solution containing bacteria and a culture medium; a sensor provided at the bottom of each of the containers and having a bacteria detection region above its top surface; and a cover for sealing the bacteria, wherein the cover is formed of a material that has a specific gravity greater than the culture medium and does not affect the detection of bacteria by the sensor, according to any one of Configurations 1 to 15.

According to such a configuration, the cover covers the area above the sensor, so that the movement of bacteria is restricted in the sealed space below the cover. In addition, since the cover does not affect the detection of bacteria by the sensor, the frequency change of the sensor due to the growth of the bacteria can be accurately measured. As described above, the state of bacteria in the bacterial solution can be accurately detected.

### Configuration 17

The bacterial inspection apparatus according to any one of Configurations 1 to 16, wherein the bacterial solution stored in the container further contains an antibacterial agent.

According to such a configuration, a drug susceptibility test of the bacteria stored in the container can be performed.

### Configuration 18

A bacterial inspection method, comprising: a step of storing a bacterial solution containing bacteria and a culture medium in a container having a sensor provided at its bottom, the sensor having a bacteria detection region above its top surface; and a step of dropping a cover into the bacterial solution by gravity to seal the bacteria with the cover and a spacer that supports the cover in a non-contact state with the top surface of the sensor.

According to such a configuration, the cover covers the area above the sensor, so that the movement of bacteria is restricted in the sealed space below the cover. In addition, since the cover 32 and the top surface 221 of the sensor 22 are separated, no pressure is applied to the top surface of the sensor 22, so the frequency change of the sensor due to the growth of the bacteria can be accurately measured. As described above, the state of bacteria in the bacterial solution can be accurately detected.

### Configuration 19

A bacterial inspection method, comprising: a step of storing a bacterial solution containing bacteria and a culture medium in a container having a sensor provided at its bottom, the sensor having a bacteria detection region above its top surface; and a step of sinking a cover into the bacterial solution to seal the bacteria, wherein the cover is formed of a material that has a specific gravity greater than the culture medium and does not affect the detection of bacteria by the sensor.

According to such a configuration, the cover covers the area above the sensor, so that the movement of bacteria is restricted in the sealed space below the cover. In addition, since the cover does not affect the detection of bacteria by the sensor, the frequency change of the sensor due to the growth of the bacteria can be accurately measured. As described above, the state of bacteria in the bacterial solution can be accurately detected.

### Configuration 20

The bacterial inspection method according to Configuration 18, further comprising a step of sedimenting the bacteria on the sensor.

According to such a configuration, all of the bacteria in the bacterial solution can be dropped onto the bottom surface of the container including the top surface of the sensor.

The embodiments disclosed this time should be considered to be illustrative in all respects and not restrictive. The scope of the present invention is indicated by the claims rather than the above description, and it is intended that all modifications within the meaning and scope equivalent to the claims are included.

### REFERENCE SIGNS LIST

1, 1a: bacterial inspection apparatus, 2: container, 4: analysis apparatus, 7: bacteria, 20, 20s: sealed space, 21: bottom part, 22: sensor, 23: bacterial solution, 24: internal space, 25: multi-well container, 26: spacer, 32, 32a, 32s: cover, 33: evaporation prevention material, 34, 34c: multi-well cover, 35: support member, 36: adhesive, 38: double-sided adhesive sheet, 42: memory, 43: input device, 44: display device, 100, 100a, 100s: container unit, 221: top surface, S: detection region.

1. A bacterial inspection apparatus, comprising:
one or more containers for storing a bacterial solution containing bacteria and a culture medium;
a sensor provided at a bottom of each of the one or more containers and having a detection region for the bacteria above a top surface thereof;
a cover for sealing the bacteria; and
a spacer for supporting the cover in a non-contact state with the top surface of the sensor.

2. The bacterial inspection apparatus according to claim 1, wherein a diameter of the cover is larger than a diameter of an opening formed by the spacer.

3. The bacterial inspection apparatus according to claim 1, wherein the spacer is arranged at the bottom.

4. The bacterial inspection apparatus according to claim 1, further comprising an evaporation prevention material provided above the bacterial solution for preventing evaporation of the bacterial solution.

5. The bacterial inspection apparatus according to claim 4, wherein the evaporation prevention material includes an oil or fat for preventing evaporation of a liquid in the bacterial solution.

6. The bacterial inspection apparatus according to claim 1, further comprising one or more positioning members above the cover for determining a position of the cover.

7. The bacterial inspection apparatus according to claim 1, wherein the bacterial inspection apparatus includes one or more support members connected to an upper part of the cover.

8. The bacterial inspection apparatus according to claim 7, wherein
the one or more containers are a plurality of containers,
the plurality of containers respectively correspond to a plurality of wells on a multi-well plate,
the one or more support members are a plurality of support members, and
the bacterial inspection apparatus further comprises a connecting member for connecting the plurality of support members.

9. The bacterial inspection apparatus according to claim 8, wherein a length of each of the plurality of support members is configured such that a sum of the length of each of the plurality of support members and a thickness of the cover is a length between an upper end of each of the plurality of containers and an upper end of the spacer.

10. The bacterial inspection apparatus according to claim 7, further comprising a lid member provided with a hole for passing each of the one or more support members up and down, respectively.

11. The bacterial inspection apparatus according to claim 1, wherein the cover seals the bacteria in a state of being sedimented on the sensor.

12. The bacterial inspection apparatus according to claim 11, wherein the cover seals the bacteria in a state of being sedimented on the sensor by a centrifuge.

13. The bacterial inspection apparatus according to claim 1, wherein the cover includes a hydrophilic membrane filter that allows the culture medium to pass through but does not allow the bacteria to pass through.

14. The bacterial inspection apparatus according to claim 1, wherein the cover is formed of a material that has a specific gravity greater than the culture medium and does not affect components of the culture medium.

15. The bacterial inspection apparatus according to claim 1, wherein the cover includes a high-molecular-weight water-absorbing polymer that absorbs moisture of the culture medium.

16. A bacterial inspection apparatus, comprising:
a container for storing a bacterial solution containing bacteria and a culture medium;
a sensor provided at a bottom of each of the containers and having a detection region for the bacteria above a top surface thereof; and
a cover for sealing the bacteria, wherein
the cover is formed of a material that has a specific gravity greater than the culture medium and does not affect detection of the bacteria by the sensor.

17. The bacterial inspection apparatus according to claim 1, wherein the bacterial solution further contains an antibacterial agent.

18. A bacterial inspection method, comprising:
a step of storing a bacterial solution containing the bacteria and a culture medium in a container having a sensor provided at a bottom thereof, the sensor having a detection region for bacteria above a top surface thereof; and
a step of dropping a cover into the bacterial solution by gravity to seal the bacteria with the cover and a spacer that supports the cover in a non-contact state with the top surface of the sensor.

19. A bacterial inspection method, comprising:
a step of storing a bacterial solution containing the bacteria and a culture medium in a container having a sensor provided at a bottom thereof, the sensor having a detection region for bacteria above a top surface thereof; and
a step of sinking a cover into the bacterial solution to seal the bacteria, wherein
the cover is formed of a material that has a specific gravity greater than the culture medium and does not affect detection of the bacteria by the sensor.

20. The bacterial inspection method according to claim 18, further comprising a step of sedimenting the bacteria on the sensor.
